(19) United States
(12) Patent Application Publication
(10) Pub. No.: US 2008/0070845 A1
(43) Pub. Date: Mar. 20, 2008

(54) Method and formulations to concurrently reduce fracture risk and insure appropriate fat-soluble vitamin supplementation when using Orlistat, an oral lipase inhibitor

(76) Inventor: Ronald V. Thompson, Fort Thomas, KY (US)

Correspondence Address: Donald N. Halgren, 35 Central Street, Manchester, MA, 01944, US

(21) Appl. No.: 11/796,982

(22) Filed: Apr. 30, 2007

Related U.S. Application Data
(63) Continuation-in-part of application No. 11/602,935, filed on Nov. 21, 2006.
Application No. 11/602,935 is a continuation-in-part of application No. 11/522,627, filed on Sep. 18, 2006, now abandoned.
Application No. 11/522,627 is a continuation-in-part of application No. 11/653,200, filed on Jan. 12, 2007, now abandoned.
Application No. 11/653,200 is a continuation-in-part of application No. 11/654,361, filed on Jan. 16, 2007.

(51) Int. Cl. A61K 31/03; A61K 31/07; A61K 31/122; A61K 31/355; A61K 31/59

(52) U.S. Cl. 514/26; 514/453; 514/725; 514/732; 514/764

(57) ABSTRACT

Orlistat is an oral lipase inhibitor that blocks the absorption of ingested fats from the small intestines, to enable proven weight loss. Fat-soluble vitamin deficiencies can be a consequence of orlistat use, especially if used for a long time without medical supervision. The present invention comprises a fat-soluble-only vitamin complex, specifically for use with orlistat, which vitamin complex contains a significantly increased amount of vitamin D, to prevent osteoporosis and to reduce fracture risk.

This invention relates to improve the use of Orlistat in human consumption, and help vitamin absorption during such use thereof. This application is a continuation-in-part of U.S. patent application Ser. No. 11/522,627, filed 18 Sep. 2006, Ser. No. 11/653,200, and 11/654,361, filed 16 Jan. 2007 each 3 incorporated herein by reference in its entirety.

### BACKGROUND OF THE INVENTION

1. Field of the Invention

2. Prior Art

Three factors in the use of Orlistat, which are inter-related but their relative associations are not understood or appreciated by the medical and scientific community.
Orlistat, an oral lipase inhibitor can induce vitamin deficiency;
Overweight, or obesity, is itself a risk factor for the developing osteoporosis and increasing fracture risk;
Current recommendations for vitamin supplementation are inadequate to prevent fracture risk and fractures.

Orlistat, an Oral Lipase Inhibitor, can Induce a Vitamin Deficiency

Webster defines a vitamin as “any group of organic substances essential in small quantities to normal metabolism, found in minute amounts in natural foodstuffs, and also produced synthetically: deficiencies of vitamins produce specific disorders”.

There are two different classifications for vitamins, fat-soluble and water-soluble vitamins. The fat-soluble vitamins are A, D, E, K and beta carotene. The water-soluble vitamins are B1, B2, B3, B6, B 12, C, G, H, K1, K2, K3, M and P.

Because Orlistat is an oral lipase inhibitor and can prevent the absorption of 25 to 35% of ingested fats, the U.S. FDA recommends multivitamin supplements while using orlistat, to prevent a vitamin deficiency of the fat soluble vitamins. In the Apr. 26, 1999 press release by the U.S. FDA, “FDA approves Orlistat for obesity”, the final paragraph of the press release states, “Because orlistat reduces the absorption of some fat-soluble vitamins and beta-carotene, patients should take a supplement that contains fat-soluble (A, D, E and K) vitamins and beta carotene”.

In product labeling for XENICAL® (120 mg orlistat) the directions for use state to ingest a multi-vitamin supplement two hours before or after ingesting XENICAL®. All currently marketed multi-vitamin supplements contain both fat-soluble and water soluble vitamins. There is currently no fat-soluble-only vitamin complex marketed.

Overweight, or Obesity is Itself a Risk Factor for Developing Osteoporosis and Increasing Fracture Risk.

In the medical publication “Risk factors for vitamin D inadequacy among women with osteoporosis: an international epidemiological study “reported in the August 2006 International Journal of Clinical Practice, Dr. Rozzoli et al concluded, “Amongst 2589 women with osteoporosis . . . recruited to determine risk factors for vitamin D inadequacy, 64% had vitamin D inadequacy. Asian ethnicity, BMI greater than or equal to 30 Kg/M2, (The definition of overweight or obesity), living in non-equitorial countries, inadequate vitamin D supplementation, poor/fair health, no education about vitamin D, skin reactivity and no recent travel to sunny areas were significant predictors.”

To better understand the defined risk factors for vitamin D inadequacy as documented by Dr. Rozzoli et al, it is first necessary to define vitamin D. Vitamin D is “any of the several fat-soluble, antirackitic (anti-ricketts) vitamins D 1, D 2, D 3, occurring in milk and fish-liver oils, especially cod and halibut; essential for the formation of normal bones and teeth.”

Vitamin D 1: a form of vitamin D obtained by ultraviolet irradiation of ergosterol.
ultraviolet irradiation—exposure to sunlight—cutaneous (skin) conversion of ergosterol into vitamin D.
ergosterol—a synthesized result of cholesterol ingestion of dietary fats
Vitamin D 2—the fat soluble predominant form of vitamin D absorbed from dietary fats—calciferol
Vitamin D 3—a form of vitamin D occurring in fish-liver oils that differs slightly in structure from vitamin D 2

Being overweight, or obese, with a BMI (Body Mass Index) of greater than or equal to 30 (Kg/m2) is itself a risk factor for vitamin D inadequacy. Therefore, a person on orlistat therapy has two risk factors for vitamin D inadequacy, the inherent risk of being overweight or obese, and the imposed risk of decreased absorption of both vitamin D 2 and D 3, and the decreased absorption or ergosterol, the substrate necessary to synthesize vitamin D 1.
Current recommendations for vitamin D supplementations are inadequate to prevent fracture risk and fractures.

The current FDA recommendation for vitamin D intake/supplementation is 400 IU per day. Multiple recently published medical articles have recommended 800 to 1000 IU of daily vitamin D intake/supplementation to reduce osteoporosis and fracture risk.

Dr. Bischoff-Ferrori et al in the May 2005 JAMA article “Fracture prevention with vitamin D supplementation “a meta-analysis of randomized controlled trials” concluded “an oral vitamin D dose of 400 IU/day is not sufficient for fracture prevention.” This conclusion was powered by multiple double blind, placebo controlled studies of almost ten thousand participants over thirty years of evaluation. Dr. R Vieth reported that four randomized, placebo controlled clinical trials demonstrated that this dose (800 IU/day of vitamin D) prevents approximately 30% of hip or on-vertebral fractures compared to placebo. “This was reported in the 2005 (37-4) Annals of Medicine article, “The role of vitamin D in the prevention of osteoporosis.”

In the journal Current Osteoporosis Research, September 2006, De M F Holick states, “Vitamin D inadequacy is pandemic in adults. Vitamin D deficiency causes osteopenia, precipitates and exacerbates osteoporosis, causes painful bone disease osteomalacia, and increases muscle weakness, which worsens the risk of falls and fractures.” In “The role of vitamin D for bone health and fracture prevention.” Dr. S. Boonen et al in “calcium and vitamin D in the prevention and treatment of osteoporosis—a clinical update” from June 2006 Journal of Internal Medicine reports “evidence based benefits are most apparent when 800 IU/day of vitamin D is complimented with a dose of 1000-1200 mg per day of elemental calcium.” Dr. Boonen also writes, “The types of individuals who should receive vitamin D supplements are:
i) patients with documented osteoporosis receiving antiresorptive or anabolic treatment
ii) patients receiving gluco corticoids
iii) individuals with or at high risk of calcium and or vitamin D insufficiencies in particular older men and women.”

In this statement, Dr. Boonen defines the high risk groups for vitamin D inadequacy and osteoporosis, but does not include individuals on Orlistat therapy. Orlistat therapy and the generalized recommendation of a multivitamin supplementation including fat-soluble vitamins by the FDA, does not recognize the specific risks of inadequate vitamin D supplementation, osteoporosis, fracture risk and fractures. To document the specific risks of orlistat, relative to osteoporosis and vitamin D deficiency, a search was accomplished in the National Library of Medicine and the National Institutes of Health data bases, (www.PUBMED.com). The specific searches

“osteoporosis risk factor for Orlistat”
No items found

“vitamin D deficiency risk factor for Orlistat”
No items found

Therefore, the medical community and the medical literature does not identify Orlistat therapy as a specific risk factor for vitamin D deficiency or osteoporosis. This is particularly important because of two future occurrences. One, Orlistat therapy is available as an over-the-counter product, and two, long term Orlistat therapy, of greater than two or three years duration, will be marketed to prevent weight regain, after the discontinuance of Orlistat. Both of these facts will amplify the increased risks of vitamin D deficiency on Orlistat therapy, and the associated increased fracture risks and fractures.

### DESCRIPTION OF THE PRESENT INVENTION

The invention comprises a fat-soluble-only vitamin complex containing vitamin A, D, E, K, and beta carotene, without water soluble vitamins, to be orally ingested during Orlistat therapy for weight loss. The fat-soluble vitamin complex will be ingested two hours before or two hours after Orlistat dosing as is currently recommended for a multivitamin with water-soluble vitamins included. In addition, the vitamin D component of the fat-soluble-only vitamin complex, would reflect a dosage of vitamin D that the recent medical literature has documented to prevent vitamin D inadequacy/insufficiency, osteoporosis risks and fracture risks.

Proposed fat-soluble vitamin complex

| | | % FDA recommended |
|---|---|---|
| Component | | of daily need |
| Vitamin A | 5000 IU | 100% |
| 41% as beta carotene | | |
| Prevents night blindness | | |
| Vitamin D | 1000 IU to 1500 IU | 250–375% |
| Prevents osteoporosis | | |
| Decreases fracture risks | | |
| Vitamin E | 45 IU | 150% |
| Anti-oxidant | | |
| Prevents premature | | |
| cell death | | |
| Vitamin K | 80 micrograms | 100% |
| Allows normal coagulation | | |

The invention thus comprises a method of treating fat-soluble vitamin deficiencies in a being under Orlistat treatment therapy, comprising the steps of: ingesting a “fat-soluble-only” vitamin supplement during the period in which the orlistat treatment occurs. The “fat-soluble-vitamin” supplement comprises vitamins A, D, E, K and beta carotene. The “fat-soluble-only” vitamin supplement is preferably taken two hours before or after Orlistat ingestion.

The invention also comprises a “fat-soluble-only” vitamin complex for the treatment of vitamin D deficiency effected by ingestion of the lipase-inhibitor treatment Orlistat, including: between 1000 IU and 1500 IU o-f vitamin D, 5000 IU of vitamin A, 45 IU of vitamin E and 80 micrograms of vitamin K.

1. A method of treating fat-soluble vitamin deficiencies in a being under Orlistat treatment therapy, comprising:
ingesting a “fat-soluble-only” vitamin supplement during the period in which the orlistat treatment occurs.

2. The method as recited in claim 1, wherein said “fat-soluble-vitamin” supplement comprises vitamins A, D, E, K and beta carotene.

3. The method as recited in claim 1, wherein said “fat-soluble-only” vitamin supplement is taken two hours before or after Orlistat ingestion.

4. A “fat-soluble-only” vitamin complex for the treatment of vitamin D deficiency effected by ingestion of the lipase-inhibitor treatment Orlistat, including: between 1000 IU and 1500 IU of vitamin D, 5000 IU of vitamin A and 45 IU of vitamin E, and 80 micrograms of vitamin K.
